(19) United States
(12) Patent Application Publication
(10) Pub. No.: US 2010/0080997 A1
(43) Pub. Date: Apr. 1, 2010

(54) Epoxy prepolymer, and epoxy resin composition, cured material, semi-cured material, prepreg and composite substrate using the epoxy prepolymer

(75) Inventors: Junichi Seki, Tokyo (JP); Kenji Tokuhisa, Tokyo (JP)

Correspondence Address: OLIFF & BERRIDGE, PLC, P.O. BOX 320850, ALEXANDRIA, VA, 22320-4850, US

(73) Assignee: TDK CORPORATION, Tokyo (JP)

(21) Appl. No.: 12/585,819

(22) Filed: Sep. 25, 2009

(30) Foreign Application Priority Data
Sep. 30, 2008 (JP) 2008-255768

(51) Int. Cl. B32B 15/092; C07D 303/12; C08G 59/06

(52) U.S. Cl. 428/418; 549/560; 528/98

(57) ABSTRACT

An epoxy prepolymer having excellent thermal conductivity is obtained by reacting an epoxy compound having a mesogenic skeleton and a trinuclear bisphenol represented by the following formula:

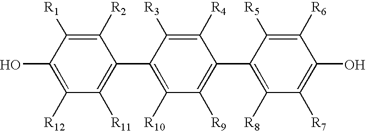

(wherein each of R₁, R₂, R₃, R₄, R₅, R₆, R₇, R₈, R₉, R₁₀, R₁₁ and R₁₂ represents a hydrogen atom or an alkyl group and each may be the same or different while at least one represents an alkyl group).

### CROSS-REFERENCES TO RELATED APPLICATIONS

The entire disclosure of Japanese Patent Application No. 2008-255768, filed on Sep. 30, 2008, is expressly incorporated herein by reference.

### BACKGROUND

1. Technical Field

The present invention relates to an epoxy prepolymer having excellent thermal conductivity. The invention also relates to an epoxy resin composition, cured material, semi-cured material, prepreg and composite substrate using the above epoxy prepolymer.

2. Related Art

Compositions containing a curing agent and an epoxy resin having a mesogenic skeleton are known as resin compositions having high thermal conductivity. For example, Japanese Patent No. 3885664 discloses a composition containing: an epoxy resin (epoxy prepolymer) obtained by reacting an epoxy compound having a specific structure including a biphenyl skeleton and a phenolic compound such as 4,4′-dihydroxybiphenyl; and an amine curing agent such as 1,5-diaminonaphthalene.

### SUMMARY

However, the thermal conductivity of the epoxy resin composition disclosed in Japanese Patent No. 3885664 is not sufficient, and there is still room for improvement.

In light of the above problem, an object of the present invention is to provide an epoxy prepolymer having excellent thermal conductivity, and an epoxy resin composition, cured material, semi-cured material, prepreg and composite substrate using the above epoxy prepolymer.

In order to solve the above problem, the present inventors carried out extensive studies, and as a result, the present inventors found that the problem can be solved by using an epoxy prepolymer obtainable by reacting an epoxy compound having a mesogenic skeleton with a trinuclear bisphenol, thereby completing the invention.

Namely, the invention provides the following:

[1] an epoxy prepolymer obtainable by reacting:
an epoxy compound having a mesogenic skeleton; and
a trinuclear bisphenol represented by the following formula:

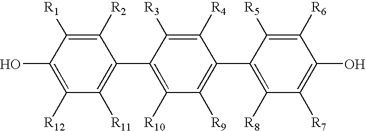

(wherein each of R₁, R₂, R₃, R₄, R₅, R₆, R₇, R₈, R₉, R₁₀, R₁₁ and R₁₂ represents a hydrogen atom or an alkyl group and each may be the same or different while at least one is an alkyl group),
[2] the epoxy prepolymer according to [1] above, wherein the trinuclear bisphenol is at least one selected from alkyl-monosubstituted trinuclear bisphenols represented by the following formula:

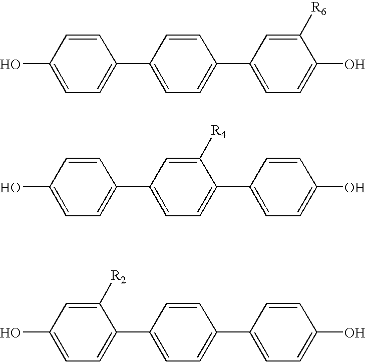

(wherein each of R₂, R₄ and R₆ represents an alkyl group),
[3] the epoxy prepolymer according to [1] or [2] above, wherein the mesogenic skeleton is represented by the following formula:

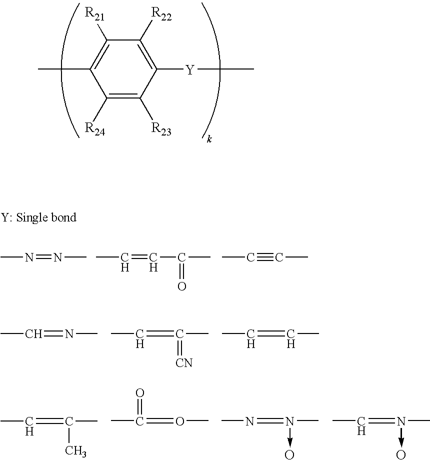

(wherein each of R₂₁, R₂₂, R₂₃ and R₂₄ represents a hydrogen atom or an alkyl group having 1 to 4 carbon atoms and each may be the same or different, and k is a number of 2 or greater),
[4] the epoxy prepolymer according to any of [1]-[3] above, wherein the epoxy compound is glycidyl ether having a biphenyl skeleton and two or more epoxy groups,
[5] an epoxy resin composition comprising:
an epoxy prepolymer obtainable by reacting an epoxy compound having a mesogenic skeleton with a trinuclear bisphenol represented by the following formula:

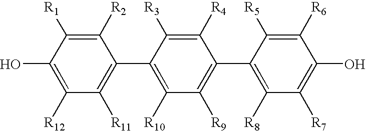

(wherein each of R₁, R₂, R₃, R₄, R₅, R₆, R₇, R₈, R₉, R₁₀, R₁₁ and R₁₂ represents a hydrogen atom or an alkyl group and each may be the same or different while at least one is an alkyl group); and
a curing agent,
   [6] a cured material obtainable by hardening an epoxy resin composition, the epoxy resin composition comprising:
an epoxy prepolymer obtainable by reacting an epoxy compound having a mesogenic skeleton with a trinuclear bisphenol represented by the following formula:

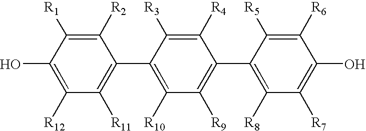

(wherein each of R₁, R₂, R₃, R₄, R₅, R₆, R₇, R₈, R₉, R₁₀, R₁₁ and R₁₂ represents a hydrogen atom or an alkyl group and each may be the same or different while at least one is an alkyl group); and
a curing agent.
   [7] the cured material according to [6] above, wherein the cured material exhibits a smectic liquid crystalline phase,
   [8] a semi-cured material obtainable by partially-hardening an epoxy resin composition, the epoxy resin composition comprising:
an epoxy prepolymer obtainable by reacting an epoxy compound having a mesogenic skeleton with a trinuclear bisphenol represented by the following formula:

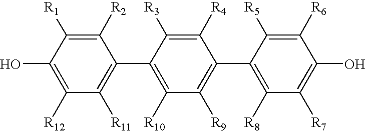

(wherein each of R₁, R₂, R₃, R₄, R₅, R₆, R₇, R₈, R₉, R₁₀, R₁₁, and R₁₂ represents a hydrogen atom or an alkyl group and each may be the same or different while at least one is an alkyl group); and
a curing agent,
   [9] a prepreg at least comprising:
a core material; and
a semi-cured material obtainable by partially-hardening an epoxy resin composition, the epoxy resin composition comprising:
   an epoxy prepolymer obtainable by reacting an epoxy compound having a mesogenic skeleton with a trinuclear bisphenol represented by the following formula:

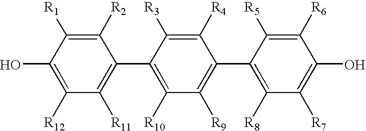

(wherein each of R₁, R₂, R₃, R₄, R₅, R₆, R₇, R₈, R₉, R₁₀, R₁₁ and R₁₂ represents a hydrogen atom or an alkyl group and each may be the same or different while at least one is an alkyl group); and
   a curing agent, and
      [10] a composite substrate comprising:
a cured material obtainable by hardening an epoxy resin composition, the epoxy resin composition comprising:
   an epoxy prepolymer obtainable by reacting an epoxy compound having a mesogenic skeleton with a trinuclear bisphenol represented by the following formula:

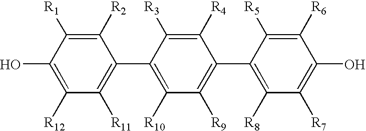

(wherein each of R₁, R₂, R₃, R₄, R₅, R₆, R₇, R₈, R₉, R₁₀, R₁₁ and R₁₂ represents a hydrogen atom or an alkyl group and each may be the same or different while at least one is an alkyl group); and
   a curing agent; and
a metal layer laminated on one surface or both surfaces of the cured material.

According to another aspect of the invention, the following are provided:

[11] an epoxy prepolymer having: a mesogenic skeleton; two or more epoxy groups; and a trinuclear skeleton represented by the following formula:

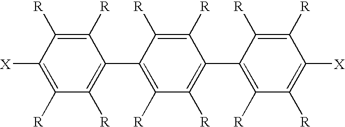

(wherein each R represents a hydrogen atom or an alkyl group and each R may be the same or different while at least one is an alkyl group, and X represents an oxygen atom), and
[12] the epoxy prepolymer according to [11] above, wherein the trinuclear skeleton is at least one selected from trinuclear skeletons represented by the following formula:

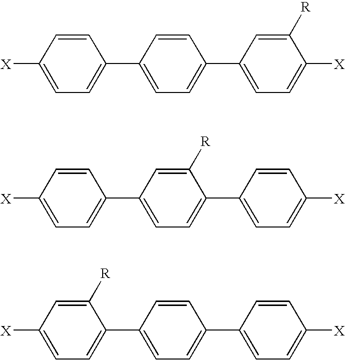

(wherein R represents an alkyl group and X represents an oxygen atom).

When measuring the properties of the epoxy resin composition containing the above epoxy prepolymer, and the cured material thereof, the present inventors found that their thermal conductivities were considerably enhanced compared to conventional products. Although the specific mechanism that brings about the above effect is still yet to be understood, a possible mechanism is as follows:

It is believed that since the above epoxy prepolymer has, in the molecule thereof, a trinuclear skeleton (triphenyl skeleton) introduced therein, the density of the aromatic ring in the resulting composition is increased compared to conventional compositions. It is also believed that a high-order structure of the stacked triphenyl skeleton and mesogenic skeleton is formed in the resulting composition, and a liquid crystalline phase having a relatively high degree of order is likely to be formed in the composition. Consequently, the cured material using the above epoxy prepolymer exhibits greatly improved thermal conductivity. Note, however, that possible mechanisms are not limited to those described above.

### BRIEF DESCRIPTION OF THE DRAWINGS

FIG. 1 is a picture of a cured material of Comparative example 1 seen under a polarizing microscope.

FIG. 2 is a picture of a cured material of Example 1 seen under a polarizing microscope.

### DESCRIPTION OF EXEMPLARY EMBODIMENTS

An embodiment of the invention is described below. The following embodiment is just an example for describing the invention, and the invention is not limited to this embodiment. The invention can be modified in various ways without departing from the gist of the invention.

The epoxy prepolymer according to this embodiment is obtainable by reacting an epoxy compound having a mesogenic skeleton with a specific trinuclear bisphenol.

Examples of the mesogenic skeleton-containing epoxy compound include, without limitation, glycidyl ethers, glycidyl esters and glycidyl amines having a mesogenic skeleton introduced therein.

The term “mesogenic skeleton” used herein refers to a partial structure that contributes to the development of a liquid crystalline property. Specific examples of the mesogenic skeleton include those represented by the following formula:

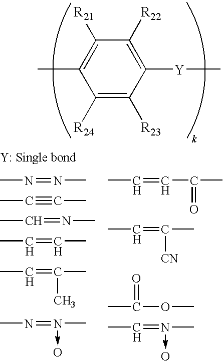

(wherein each of R₂₁, R₂₂, R₂₃ and R₂₄ represents a hydrogen atom or an alkyl group having 1 to 4 carbon atoms and each may be the same or different, and k is a number of 2 or greater).

Of these, the mesogenic skeleton is preferably one represented by the following formula:

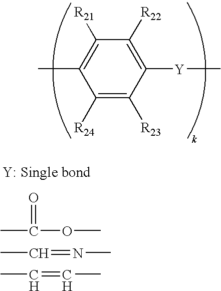

(wherein each of R₂₁, R₂₂, R₂₃ and R₂₄ represents a hydrogen atom or an alkyl group having 1 to 4 carbon atoms and each may be the same or different, and k is a number of 2 or greater).

In particular, in order to further improve the thermal conductivity, it is preferable that the mesogenic skeleton-containing epoxy compound has, in the molecule thereof, a biphenyl skeleton, and more specifically, it is particularly preferable that the epoxy compound is a glycidyl ether having, in the molecule thereof, a biphenyl skeleton and two or more epoxy groups (e.g., biphenyl glycidyl ether and tetramethylbiphenyl glycidyl ether).

The trinuclear bisphenol is a phenolic compound having an alkyl-substituted triphenyl skeleton, more specifically, 4,4″-dihydroxy-p-triphenyl, represented by the following formula:

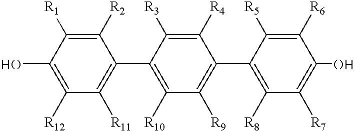

(wherein each of R₁, R₂, R₃, R₄, R₅, R₆, R₇, R₈, R₉, R₁₀, R₁₁ and R₁₂ represents a hydrogen atom or an alkyl group and each may be the same or different while at least one is an alkyl group).

In particular, the trinuclear bisphenol is preferably at least one selected from alkyl-monosubstituted trinuclear bisphenols represented by the following formula:

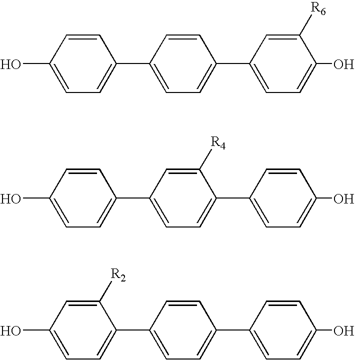

(wherein each of R₂, R₄ and R₆ represents an alkyl group),
from the viewpoint that those trinuclear bisphenols are easy to synthesize and can be mass-produced at a low cost.

The trinuclear bisphenol has at least one alkyl group. Such alkyl-substituted products are fairly easy to synthesize and have excellent solubility in a solvent, compared to non-substituted products. Accordingly, by using the above trinuclear bisphenol, productivity and economic efficiency can be further enhanced, and handleability can also be improved.

There are no particular limitations on the alkyl group referred to herein, and it may be any of a linear alkyl group, a branched alkyl group and a cyclic alkyl group. The alkyl group is preferably a linear alkyl group. Also, there are no particular limitations on the number of carbon atoms in the alkyl group, and the carbon number is preferably 1 to 8, more preferably 1 to 6, and still more preferably 1 to 4. There are no particular limitations on the number of alkyl substitutions, and the number is preferably 1 to 6.

The mesogenic skeleton-containing epoxy compound and the trinuclear bisphenol are normally reacted in a solvent, in the presence of a catalyst if necessary, while being heated. As a result, epoxy groups and phenolic hydroxyl groups are reacted, resulting in the formation of an epoxy prepolymer having a mesogenic skeleton, a trinuclear skeleton, and two or more epoxy groups. The epoxy prepolymer thus obtained has a high-order structure of the arrayed mesogenic skeleton and trinuclear skeleton, and it is believed that this high-order structure brings about the development of high thermal conductivity.

There are no particular limitations on the solvent used herein as long as the mesogenic skeleton-containing epoxy compound and the trinuclear bisphenol can be dissolved or dispersed in the solvent, and examples include methyl ethyl ketone, methyl isobutyl ketone, dimethyl formamide, propylene glycol monomethyl ether, and a solvent mixture thereof.

There are no particular limitations on the amount of the mesogenic skeleton-containing epoxy compound and the amount of the trinuclear bisphenol to be reacted, and the amounts may arbitrarily be determined so as to result in a particular equivalent ratio according to the number of epoxy groups contained in the epoxy compound and the number of phenolic hydroxyl groups contained in the trinuclear bisphenol. Typically, when using a difunctional epoxy compound and a trinuclear bisphenol, the equivalent ratio is generally set to around 0.5. Note that the mesogenic skeleton-containing epoxy compound may be used alone or in combination of two or more types thereof, and that the trinuclear bisphenol may also be used alone or in combination of two or more types thereof. There are no particular limitations on the temperature conditions, and generally, the temperature may arbitrarily be determined within the range of from 120 to 200° C.

The epoxy resin composition according to this embodiment at least contains the above-described epoxy prepolymer and a curing agent. Examples of the curing agent used herein include amine compounds, imidazole compounds, and derivatives thereof, and known curing agents in the art may be arbitrarily used.

In order to achieve further enhanced thermal conductivity, it is preferable to use a curing agent having a biphenylaralkyl skeleton. Examples of the above curing agent include, without limitation, polyfunctional phenols and aromatic amines having a biphenylaralkyl skeleton introduced therein.

Specific examples of the biphenylaralkyl skeleton include those represented by the following formula:

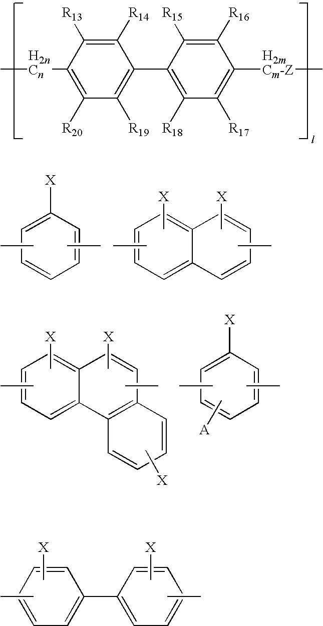

(wherein each of R₁₃, R₁₄, R₁₅, R₁₆, R₁₇, R₁₈, R₁₉ and R₂₀ represents a hydrogen atom or a monovalent alkyl group and each may be the same or different; each X represents a hydrogen atom or a hydroxyl group and each may be the same or different; A represents a hydroxyl group or a monovalent alkyl group; I, as mean value, is a number greater than 1; n and m are each integers of 1 or greater; and Z represents a group having at least one hydroxyl group).

In particular, from the viewpoint of the facility of industrial synthesis, the biphenylaralkyl skeleton-containing curing agent is preferably one represented by the following formula:

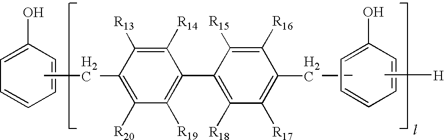

(wherein each of R₁₃, R₁₄, R₁₅, R₁₆, R₁₇, R₁₈, R₁₉ and R₂₀ represents a hydrogen atom or a monovalent alkyl group and each may be the same or different, and I, as mean value, is a number greater than 1)

The biphenylaralkyl skeleton-containing curing agent is preferably an amorphous curing agent from the viewpoint of handleability, and the curing agent is preferably a curing agent having no melting point from other viewpoints.

The biphenylaralkyl skeleton-containing curing agent preferably has a softening point of 110° C. or lower, more preferably 100° C. or lower, still more preferably 90° C. or lower, and particularly preferably 80° C. or lower, in order to exhibit good moldability at a relatively low temperature and improve handleability.

There are no particular limitations on the mixture ratio between the mesogenic skeleton-containing epoxy compound and the biphenylaralkyl skeleton-containing curing agent, and the biphenylaralkyl skeleton-containing curing agent is preferably used in an amount of from 5 to 40 parts by mass, more preferably from 10 to 30 parts by mass, based on 100 parts by mass of the mesogenic skeleton-containing epoxy compound, in terms of the solid content thereof. If the mesogenic skeleton-containing epoxy compound or the biphenylaralkyl skeleton-containing curing agent is used in excess, the heat-resistance of a hardened resin material tends to be reduced.

Note that the epoxy resin composition may contain two or more of the above epoxy prepolymer, or may contain an epoxy compound or an epoxy prepolymer other than the above epoxy prepolymer, for example, an epoxy compound not having a mesogenic skeleton. Also, the curing agent may be used in combination of two or more thereof.

The epoxy resin composition is normally used in a state of being dissolved or dispersed homogenously in a solvent. There are no particular limitations on the solvent used herein as long as the above epoxy compound and curing agent can be dissolved or dispersed in the solvent, and examples of the solvent include: methyl ethyl ketone, methyl cellosolve, methyl isobutyl ketone, dimethyl formamide, propylene glycol monomethyl ether, toluene, xylene, acetone, and a solvent mixture thereof.

The epoxy composition may contain, if necessary, components other than the above described two components. Examples of such additional components include curing catalysts (curing accelerators) such as phosphines and imidazoles (2-ethyl-4-methyl imidazole), coupling agents such as silane coupling agents and titanate coupling agents, inorganic fillers such as alumina and silica, fibers such as glass fibers and ceramics fibers, woven cloth, nonwoven cloth, flame retardants such as halogen and phosphorous compounds, diluents, plasticizers and lubricants, and they may be arbitrarily selected from those known in the art.

By heating and drying the above epoxy resin composition, a semi-cured material of the epoxy resin composition, i.e., the epoxy resin composition in a so-called B-stage state can be obtained. There are no particular limitations on the process for producing the semi-cured material, and a common process may be used. Typically, a process of heating and drying the epoxy resin composition put and held in a mold of a specific shape, and a process of applying the epoxy resin composition onto a resin film such as PET or a support such as a metal plate and then heating and drying the epoxy resin composition may be used. The epoxy resin composition according to this embodiment can be partially-hardened, for example, under the conditions of about 1 to 120 minutes at a temperature of 60 to 150° C., and the conditions are preferably about 10 to 90 minutes at a temperature of 70 to 120° C. Since the epoxy resin composition according to this embodiment can be treated at a relatively low temperature, it is superior to conventional products.

By heating the above epoxy resin composition or semi-cured material thereof until the hardening reaction has progressed sufficiently, a cured material can be obtained. There are no particular limitations on the process for producing the cured material, and a common process may be used. The heating conditions are typically about 1 to 300 minutes at 100 to 200° C. The production of the cured material may be performed under pressure.

The thermal conductivity of the cured material thus obtained is preferably 0.35 (W/m*K) or greater, more preferably 0.38 (W/m*K) or greater, and still more preferably 0.40 (W/m*K) or greater.

The above cured material preferably exhibits a smectic liquid crystalline phase. When using an epoxy compound having a mesogenic skeleton, the cured material thereof is normally observed under a polarizing microscope as exhibiting a nematic liquid crystalline phase with a schlieren texture. However, when using an epoxy prepolymer obtainable by reacting an epoxy compound having a mesogenic skeleton with a trinuclear bisphenol, the cured material thereof surprisingly tends to be observed under a polarizing microscope as exhibiting a smectic liquid crystalline phase, and such products exhibiting a smectic liquid crystalline phase tend to be particularly excellent in thermal conductivity.

A semi-cured material is formed by partially-hardening the above epoxy resin composition. By impregnating a core material with the above epoxy resin composition, for example, by applying the epoxy resin composition to the core material or by immersing the core material in the epoxy resin composition, and thereafter drying and partially-hardening the epoxy resin composition, a prepreg can be prepared. Also, by laminating the above prepreg and a metal layer such as a metal plate or metal foil, and then hardening, and if necessary, heating and pressing the laminated product, a metal-clad laminate (composite substrate) can be prepared. The preparation methods are not limited to those described above.

The core material used for the prepreg may be arbitrarily selected from various known materials. For example, glass fiber, metal fiber, natural fiber, synthesized fiber, and woven or nonwoven cloth formed, for example, of synthesized fiber such as polyester fiber or polyamide fiber may be used, although the applicable materials are not limited to the above. These core materials may be used alone or in combination of two or more thereof. There are no particular limitations on the thickness of the core material, and the thickness may be arbitrarily determined in accordance with the thickness of the prepreg or the laminate, a desired mechanical strength and size stability, etc. The thickness is normally within the range of about 0.03 to 0.20 mm.

The metal layer used for the composite substrate may be arbitrarily selected from various known materials. For example, metal plates and metal foil of Cu, Al, etc., may be used, although the applicable materials are not limited to the above. There are no particular limitations on the thickness of the metal layer, and the thickness is normally within the range of about 3 to 150 μm.

### EXAMPLES

The embodiment of the invention is more specifically described referring to the Synthesis examples, Examples, and Comparative examples below. The terms “parts” and “%” used below indicate “parts by mass” and “% by mass” respectively.

### Epoxy Prepolymer and Epoxy Resin Composition

### Example 1

50 parts by mass of a difunctional crystalline epoxy compound represented by the formula shown below (trade name: YL6121H, product of Japan Epoxy Resins Co., Ltd., epoxy equivalent: 175) and 21.17 parts by mass of a trinuclear bisphenol (4,4″-dihydroxy-3-methyl-p-triphenyl, abbreviated as DHTP-M, equivalent: 138) were placed in a three-mouth flask (equivalent ratio: 0.5), and 166 parts by mass of methyl ethyl ketone were further added so that the solid content in the resulting mixture was 30% by mass. The resulting mixture was stirred after setting the temperature so as to bring the mixture under reflux. Upon observing the flask having reflux inside, the stirring reaction was carried out for twelve hours. After that, the mixture was cooled to room temperature, and as a result, an epoxy prepolymer of Example 1 was synthesized. In this epoxy prepolymer solution, 14.07 parts by mass (equivalent ratio: 0.5) of a biphenylaralkyl curing agent represented by the formula shown below (trade name: HE200C, product of Air Water Inc., equivalent: 212, average n=1.2, softening point=75° C.) and 0.1825 part by mass of a curing catalyst (2-ethyl-4-methyl imidazole, abbreviated as 2E4Mz, product of Shikoku Chemicals Corporation) were mixed and dispersed homogeneously, resulting in the preparation of an epoxy resin composition of Example 1.

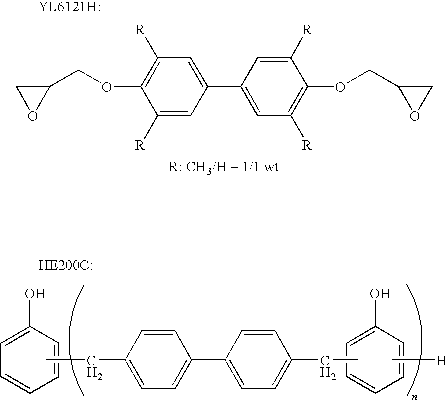

### Example 2

In the same manner as Example 1 other than replacing the curing agent with a biphenylaralkyl curing agent represented by the formula shown below (trade name: MEH7851, product of Meiwa Plastic Industries, Ltd., equivalent: 212, average n=10, softening point=73° C.), an epoxy resin composition of Example 2 was prepared.

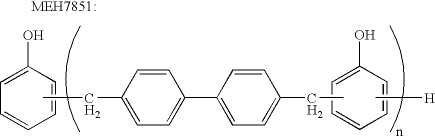

### Comparative Example 1

100 parts by mass of a difunctional crystalline epoxy compound (trade name: YL6121H, product of Japan Epoxy Resins Co., Ltd., epoxy equivalent: 175) and 28.53 parts by mass of dihydroxy biphenyl (DHBP) represented by the formula shown below were placed in a three-mouth flask (equivalent: 93), and 128.53 parts by mass of methyl ethyl ketone were further added so that the solid content in the resulting mixture was 50% by mass. The resulting mixture was stirred after setting the temperature so as to bring the mixture under reflux. After observing that YL6121H and DHBP were dissolved, the stirring reaction was carried out for twelve hours. After that, the mixture was cooled to room temperature, and as a result, an epoxy prepolymer of Comparative Example 1 was synthesized. In this epoxy prepolymer solution, 28.15 parts by mass (equivalent ratio: 0.5) of a biphenylaralkyl curing agent (trade name: HE200C, product of Air Water Inc., equivalent: 212, average n=1.2, softening point=75° C.) and 0.3355 part by mass of a curing catalyst (2-ethyl-4-methyl imidazole, abbreviated as 2E4Mz, product of Shikoku Chemicals Corporation) were mixed and dispersed homogeneously, resulting in the preparation of an epoxy resin composition of Comparative Example 1.

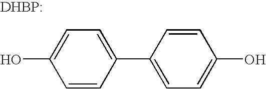

### Comparative Example 2

In the same manner as Comparative Example 1 other than replacing the curing agent with another biphenylaralkyl curing agent (trade name: MEH7851, product of Meiwa Plastic Industries, Ltd., equivalent: 212, average n=10, softening point=73° C.), an epoxy resin composition of Comparative Example 2 was prepared.

### <Semi-Cured Material and Cured Material>

The above obtained epoxy resin compositions of Examples 1 and 2 were applied onto a PET film, and dried at 100° C. for 15 minutes to evaporate the solvent and bring the epoxy resin compositions in a B-stage state, and as a result, semi-cured materials of Examples 1 and 2 were respectively prepared. The obtained partially-hardened B-stage materials were placed in a specific mold, and pressed for 15 minutes at 185° C. and 25 MPa using a hand-pressing machine. Then, by carrying out heat treatment at 185° C. for three hours, cured materials of Examples 1 and 2 were respectively prepared.

The above obtained epoxy resin compositions of Comparative Examples 1 and 2 were applied onto a PET film, and dried at 100° C. for 30 minutes to evaporate the solvent and bring the epoxy resin compositions in a B-stage state, and as a result, semi-cured materials of Comparative Examples 1 and 2 were respectively prepared. The obtained partially-hardened B-stage materials were placed in a specific mold, and pressed for 15 minutes at 185° C. and 25 MPa using a hand-pressing machine. Then, by carrying out heat treatment at 185° C. for three hours, cured materials of Comparative Examples 1 and 2 were respectively prepared.

The evaluation results on the properties of the epoxy resin compositions of Examples 1 and 2 and Comparative Examples 1 and 2, and cured materials thereof are shown in Table 1.

TABLE 1

| Epoxy resin composition | | Example | | Comparative Example | |
|---|---|---|---|---|---|
| (parts by mass) | | 1 | 2 | 1 | 2 |
| Epoxy compound | YL6121H | 50 | 50 | 100 | 100 |
| Trinuclear | DHBP | — | — | 28.53 | 28.53 |
| bisphenol | DHTP-M | 21.17 | 21.17 | — | — |
| Curing agent | HE200C | 14.07 | — | 28.15 | — |
| | MEH7851 | — | 14.07 | — | 28.15 |
| Curing catalyst | 2E4Mz | 0.1825 | 0.1825 | 0.3355 | 0.3355 |

| | | | | |
|---|---|---|---|---|
| Thermal conductivity | 0.43 | 0.40 | 0.32 | 0.30 |
| (W/m * K) | | | | |

The evaluation was performed in the following manner:

### Thermal Conductivity Measurement of Cured Materials

A cured material was stamped out in the form of a 1 mmø disk to prepare a measurement sample. The obtained measurement sample was subjected to the thermal conductivity measurement using a thermal conductivity measurement apparatus (trade name: TC Series, product of ULVAC-RIKO, Inc.). The specific heat was measured based on DSC using sapphire as a standard sample, and the thermal conductivity was calculated by assigning the measured value to equation (1) below.

λ=α*Cp*r (1)
α: thermal diffusivity
Cp: specific heat
r: density

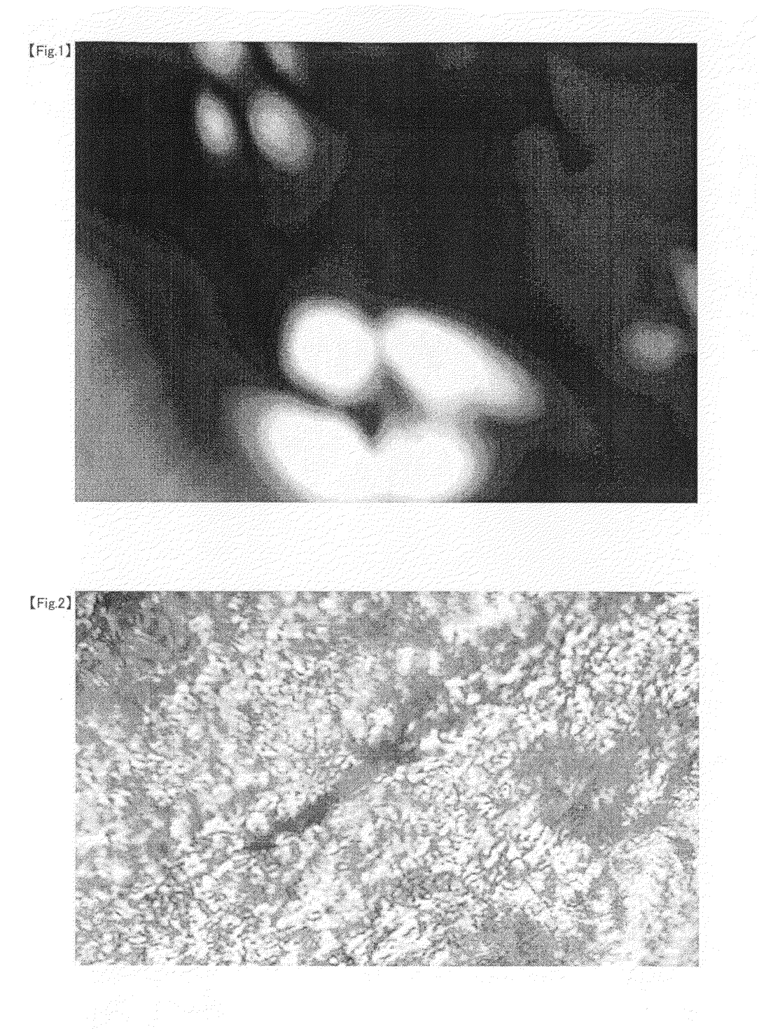

In addition, FIGS. 1 and 2 show the results of observing the cured materials of Comparative Example 1 and Example 1 under a polarizing microscope (trade name: OPTIHOT, product of Nicon Corporation) under the condition of crossed Nicols. As can be seen from FIGS. 1 and 2, nematic liquid crystal with a schlieren texture was observed in the cured material of Comparative Example 1, whereas smectic liquid crystal was observed in the cured material of Example 1. This clearly shows that a liquid crystalline phase having a relatively high degree of order was formed in the epoxy prepolymer obtained by using a trinuclear bisphenol, compared to the epoxy prepolymer obtained without using such a trinuclear bisphenol.

As described above, the epoxy prepolymer according to the invention, and epoxy resin compositions, cured materials, semi-cured materials, prepregs and composite substrates obtained using the above epoxy prepolymer are excellent in thermal conductivity, and accordingly, in the field of electronic materials which require high thermal conductivity, they can be widely and effectively used for electronic parts as well as modules such as substrates with electronic parts, cooling sheets, and insulating materials.

According to the invention, an epoxy prepolymer having excellent thermal conductivity can be obtained, and consequently, epoxy resin compositions, cured materials, semi-cured materials, prepregs and composite substrates that are excellent in thermal conductivity can be easily provided at a low cost, resulting in improved productivity and economic efficiency.

1-10. (canceled)

11. An epoxy prepolymer obtainable by reacting:
an epoxy compound having a mesogenic skeleton; and
a trinuclear bisphenol represented by the following formula: 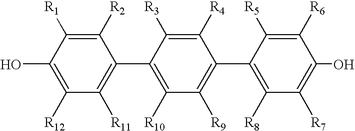
(wherein each of R₁, R₂, R₃, R₄, R₅, R₆, R₇, R₈, R₉, R₁₀, R₁₁ and R₁₂ represents a hydrogen atom or an alkyl group and each may be the same or different while at least one is an alkyl group).

12. The epoxy prepolymer according to claim 11, wherein the trinuclear bisphenol is at least one selected from alkyl-monosubstituted trinuclear bisphenols represented by the following formula: 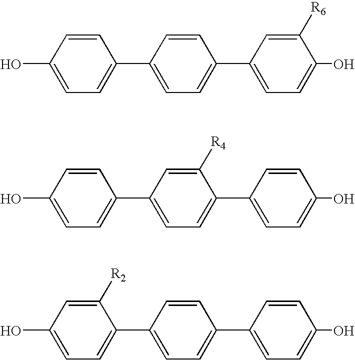
(wherein each of R₂, R₄ and R₆ represents an alkyl group).

13. The epoxy prepolymer according to claim 11, wherein the mesogenic skeleton is represented by the following formula: 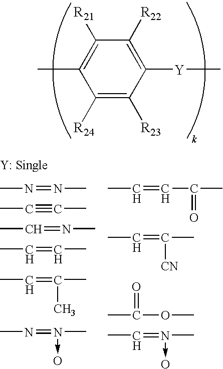
(wherein each of R₂₁, R₂₂, R₂₃ and R₂₄ represents a hydrogen atom or an alkyl group having 1 to 4 carbon atoms and each may be the same or different, and k is a number of 2 or greater).

14. The epoxy prepolymer according to claim 12, wherein the mesogenic skeleton is represented by the following formula: 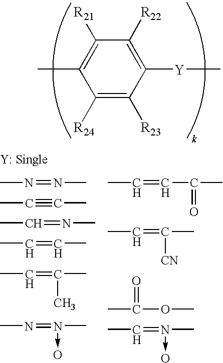
(wherein each of R₂₁, R₂₂, R₂₃ and R₂₄ represents a hydrogen atom or an alkyl group having 1 to 4 carbon atoms and each may be the same or different, and k is a number of 2 or greater).

15. The epoxy prepolymer according to claim 11, wherein the epoxy compound is glycidyl ether having a biphenyl skeleton and two or more epoxy groups.

16. The epoxy prepolymer according to claim 12, wherein the epoxy compound is glycidyl ether having a biphenyl skeleton and two or more epoxy groups.

17. The epoxy prepolymer according to claim 13, wherein the epoxy compound is glycidyl ether having a biphenyl skeleton and two or more epoxy groups.

18. The epoxy prepolymer according to claim 14, wherein the epoxy compound is glycidyl ether having a biphenyl skeleton and two or more epoxy groups.

19. An epoxy resin composition comprising:
an epoxy prepolymer obtainable by reacting an epoxy compound having a mesogenic skeleton with a trinuclear bisphenol represented by the following formula: 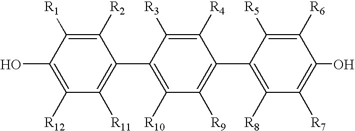
(wherein each of R₁, R₂, R₃, R₄, R₅, R₆, R₇, R₈, R₉, R₁₀, R₁₁ and R₁₂ represents a hydrogen atom or an alkyl group and each may be the same or different while at least one is an alkyl group); and
a curing agent.

20. A cured material obtainable by hardening an epoxy resin composition according to claim 19.

21. The cured material according to claim 20, wherein the cured material exhibits a smectic liquid crystalline phase.

22. A semi-cured material obtainable by partially-hardening an epoxy resin composition according to claim 19.

23. A prepreg comprising:
a core material; and
a semi-cured material according to claim 22.

24. A composite substrate comprising:
a cured material obtainable by hardening an epoxy resin composition according to claim 19; and
a metal layer laminated on one surface or both surfaces of the cured material.
